(19) United States
(12) Patent Application Publication
(10) Pub. No.: US 2006/0058822 A1
(43) Pub. Date: Mar. 16, 2006

(54) Blade and blade carrier suitable therefor

(75) Inventors: Christian Rathjen, Bremen (DE); Thomas Hunkeler, Kerzers (CH)

Correspondence Address: OLIFF & BERRIDGE, PLC, P.O. BOX 19928, ALEXANDRIA, VA, 22320, US

(73) Assignee: SIS AG, Surgical Instrument Systems, Port (CH)

(21) Appl. No.: 11/227,181

(22) Filed: Sep. 16, 2005

Related U.S. Application Data
(60) Provisional application No. 60/610,190, filed on Sep. 16, 2004.

(30) Foreign Application Priority Data
Sep. 16, 2004 (EP) 04 405 585.3

(51) Int. Cl. A61F 9/00

(52) U.S. Cl. 606/166

(57) ABSTRACT

In a blade (1) for cutting away an epithelial flap from a Bowman's membrane (31), the blade tip (11) is designed in such a way that, in the state in which the blade tip (11) is inserted between epithelium (2) and Bowman's membrane (31), said blade tip (11) spreads open a clearance space between the epithelium (2) and the Bowman's membrane (31). The clearance space thus held open results in a pre-tensioning of connections between the epithelium (2) and the Bowman's membrane (31). The cutting edge of the blade tip (11) is rounded and has a thickness which corresponds at least substantially to the thickness of the epithelium (2). The blade (1) is held removably in a blade holder (8) in such a way that the blade tip (11), in relation to the cutting plane (9), has an application clearance angle in a range of from 10° to 30°. Drive means (82, 83) of the blade holder (8) move the blade (1) in the advance direction (7) and set the cutting edge of the blade (1) in a lateral oscillating movement perpendicular to the advance direction (7) in the cutting plane (9), the advance travel being negligible compared to the cumulative lateral travel of the cutting edge resulting from the oscillation movement. As a result of the pre-tensioning and of the alternating load caused by the oscillating movement, the connection between the epithelium (2) and the Bowman's membrane (31) can be separated without forward pressure having to be exerted on the epithelial cells to do this, with the result that intercellular connections of the epithelium (2) are not impaired.

### TECHNICAL FIELD

The present invention relates to a blade and to a blade holder suitable for same. The present invention relates in particular to a blade for cutting away an epithelial flap from a Bowman's membrane, said blade comprising a blade shaft and, connected to the latter, a blade tip with cutting edge, and also to a blade holder suitable for this blade.

### PRIOR ART

Conditions causing impaired vision, such as myopia (near-sightedness), hyperopia (far-sightedness or long-sightedness) or astigmatism can nowadays be permanently corrected by refractive surgery. Refractive surgery involves surgical procedures on the eye which modify the optical refractive power of the eye with the aim of bringing it as close as possible to a desired value. One of the most important methods used in refractive surgery is laser-assisted in situ keratomileusis (LASIK) in which the interior of the cornea is removed using a computer-controlled excimer laser, after a corneal flap has first been partly detached and folded aside. To produce the corneal flap, mechanical microkeratomes are generally used in which a driven knife cuts the corneal flap.

Patent application WO 2004/052254 describes a separator element and a method for separating the epithelium of the cornea from the underlying Bowman's membrane. The separator element comprises a blade which is not so sharp as to penetrate into the Bowman's membrane. According to WO 2004/052254, the epithelial cells are pressed aside by the separating edge, preferably without damaging the intercellular connections of the epithelium. The fibrils which connect the epithelium to the Bowman's membrane are divided by the separating edge without incision of the Bowman's membrane. According to WO 2004/052254, the separating edge has a thickness substantially below the thickness of the epithelium, preferably a thickness of 15 μm.

Patent application US 2003/0018348 describes a device for separating the epithelium of the cornea mechanically from an eye. The device according to US 2003/0018348 comprises a separator element with a cutting edge for removing the epithelium during the movement of the separator element across the eye. According to US 2003/0018348, the cutting edge has a thickness which is at least as thick as a single layer of epithelial cells, but less thick than the thickness of the epithelium. The cutting edge according to US 2003/0018348 preferably has a thickness of two to three cell layers of the epithelium. According to US 2003/0018348, the epithelial cells are pressed aside during the movement of the separator element across the eye, without the intercellular connections of the epithelium being damaged.

The known methods and devices for removing the epithelium from an eye have the disadvantage that they sever the connections between the Bowman's membrane and epithelium as a result of the epithelial cells being pressed aside as the separator element is advanced. With forwardly directed pressure, however, the epithelium is deformed such that shearing forces are generated within the epithelium and may destroy the connections between the epithelial cells. With forwardly directed pressure, there is therefore a danger that, even with careful application, connections between individual epithelial cells, and thus the epithelium itself, will be destroyed, for example torn apart, which has a negative impact on the fusion of the epithelial tissue to the Bowman's membrane during reconstruction of the cornea and thus may have a negative impact on the success of refractive surgery.

### DISCLOSURE OF THE INVENTION

It is an object of the present invention to make available a blade for cutting away an epithelial flap from a Bowman's membrane, and also a blade holder suitable for this, which blade and blade holder do not have the disadvantages of the known devices. It is in particular an object-of the present invention to make available a blade for cutting away an epithelial flap from a Bowman's membrane, and also a blade holder suitable for this, which blade and blade holder make it possible to release the connections between the Bowman's membrane and the epithelium without having to exert direct mechanical forward pressure on the epithelial cells in order to do so.

According to the present invention, these objects are achieved in particular by the features of the independent claims. Further advantageous embodiments are also set out in the dependent claims and in the description.

The abovementioned objects are achieved by the present invention in particular by the fact that, in the blade for cutting away an epithelial flap from a Bowman's membrane, comprising a blade shaft and, connected to the latter, a blade tip with cutting edge, the blade tip is designed in such a way that, in the state in which the blade tip is inserted between epithelium and Bowman's membrane, said blade tip spreads open a clearance space between the epithelium and the Bowman's membrane, which clearance space results in a pre-tensioning of connections between the epithelium and the Bowman's membrane. The clearance space produced by insertion of the blade tip between epithelium and Bowman's membrane has a substantially wedge-shaped cross section which extends perpendicular to the cutting edge and which narrows from the cutting edge to an apex where the epithelium and the Bowman's membrane are still connected to one another. The creation of pre-tensioning on the connections between the epithelium and the Bowman's membrane has the advantage that these connections do not have to be destroyed by forward pressure on the epithelial cells, such that the epithelium is not adversely deformed, and intercellular connections of the epithelium are therefore not impaired. Because of the pre-tensioning of the connections between the Bowman's membrane and the epithelium, the advance of the cutting edge in the cutting plane can in fact be set such that it is negligible compared to lateral oscillating movements of the cutting edge in the cutting plane perpendicular to the advance direction. The already separated part of the epithelium lying on the blade tip upper face is entrained by frictional forces in the lateral oscillating movements, which leads to continuous alternate loading of the pre-tensioned connections. As a result of the alternate loading of the pre-tensioned connections, fibres of the connections gradually tear, finally resulting in a kind of fatigue fracture of the connection.

The blade preferably has an edgeless and smooth contact surface, and, in the state in which the blade tip is inserted between epithelium and Bowman's membrane, this contact surface presses at least partially on the Bowman's membrane. The edgeless and smooth configuration of the contact surface ensures that the blade, particularly during the lateral oscillating movements in the cutting plane perpendicular to the advance direction, does not act with an abrasion effect on the Bowman's membrane and damage the latter. On the other hand, pressure is exerted on the Bowman's membrane by the contact surface, and, in this way, the pre-tensioning of the connections between epithelium and Bowman's membrane, produced by the clearance space, is built up and stabilized.

In one embodiment variant, the contact surface arranged on the blade tip is angled away from the underside of the blade shaft in such a way that the blade tip has a clearance angle. The fact that the contact surface is angled away from the underside of the blade shaft means that, during application of the blade, the blade shaft has a greater application clearance angle than does the blade tip, with the result that the blade comes to lie on the Bowman's membrane via (part) of the contact surface of the blade tip, not via the blade shaft. In this way, unnecessary additional frictional forces on the Bowman's membrane are avoided, and therefore, on the one hand, the Bowman's membrane and, on the other hand, the drive means for the advance movement and lateral oscillation of the blade are not unnecessarily loaded.

The cutting edge preferably has a thickness which corresponds at least substantially to the thickness of the epithelium. The cutting edge has, for example, a thickness of 60 μm or more. A cutting edge thickness which has at least the thickness of the epithelium or a greater thickness contributes decisively to generating the clearance space between the epithelium and the Bowman's membrane and thus to pre-tensioning the connections between the epithelium and the Bowman's membrane. In addition, by selecting a cutting edge of this thickness, the risk of penetration into the Bowman's membrane is further reduced.

The cutting edge is preferably rounded. The cutting edge preferably has a rounding with a radius of at least half the thickness of the epithelium or a greater radius. The radius of the rounding of the cutting edge is, for example, 30 μm or more. A rounded cutting edge or blade tip has the advantage that it is easy and inexpensive to produce, for example it can be produced as one piece made from plastic by an injection moulding operation. The rounded cutting edge further reduces the risk of penetration into the Bowman's membrane and helps the blade tip slide across the Bowman's membrane. The rounded cutting edge or blade tip also facilitates the opening of the clearance space between the epithelium and the Bowman's membrane, since a rounded cutting edge or blade tip with smooth surface can be introduced without high frictional forces between epithelium and Bowman's membrane, and in this way the pre-tensioning of the connections between the epithelium and the Bowman's membrane can be more easily built up.

In one embodiment variant, the blade tip has a smoothly configured blade tip upper face and, in the state in which the blade tip is inserted between epithelium and Bowman's membrane, the epithelial flap lies on this blade tip upper face, and the blade tip upper face has a recess, in which recess an upward wall of a blade holder can be received in such a way that the transition from the blade tip upper face to the upward wall does not form an obstacle to the epithelial flap sliding up it during continued cutting. In the state of insertion into the blade holder, the cut-away epithelial flap, during the advance movement of the cutting edge, is thus able to slide unimpeded across the blade tip upper face and across the upward wall of the blade holder.

In addition to the blade described above, the present invention also relates to a blade holder which is designed to receive the blade in a removable manner. The above-described blade can, for example, be connected removably to the blade holder by means of a plug connector or a clamping mechanism. The blade holder preferably comprises known suction means for fixing the blade holder on an eye.

The blade holder preferably comprises first drive means for moving the blade in an advance direction. In addition, the blade holder comprises second drive means for setting the cutting edge of the blade in a lateral oscillating movement perpendicular to the advance direction in a cutting plane, a cumulative lateral travel of the cutting edge obtained from the oscillating movement being considerably greater than a travel of the cutting edge arising within the same time period in the advance direction. For example, with a lateral oscillating movement at a frequency of 150 Hz, there is a maximum lateral speed of the cutting edge of ca. 1.5 m/sec, which, in the case of an advance speed of 1.5 mm/sec, gives an almost thousand times greater cumulative travel of the cutting edge in the lateral direction than in the advance direction. That is to say, compared to the oscillating movement, the advance movement is negligible. Consequently, the pre-tensioned connections between the Bowman's membrane and the epithelium are built up, as has been described above, on account of the oscillating movement of the blade tip, without the epithelial cells being impaired by forward pressure. In the reconstruction of the cornea, the gentle treatment of the epithelial cells has an advantageous effect on the fusion of the epithelial tissue to the Bowman's membrane, which in turn has a positive influence on the success of the refractive surgery.

In one embodiment variant, the second drive means are designed to generate the lateral oscillating movement with an oscillation stroke of the cutting edge in the range of from 2 mm to 4 mm, preferably 3 mm (oscillation amplitude of 1 mm to 2 mm, preferably ca. 1.5 mm).

In one embodiment variant, the second drive means are designed to generate the lateral oscillating movement with a frequency in the range of from 150 Hz to 200 Hz, preferably 180 Hz. At this frequency of the oscillating movement, a good cutting quality can be achieved with smooth and undamaged surfaces of the exposed Bowman's membrane and of the underside of the cut-away epithelial flap.

The blade holder is preferably designed to receive the blade in such a way that the blade tip has an application clearance angle in a range of from 10° to 30° in relation to the cutting plane. The blade holder is, for example, designed to receive the blade in such a way that the blade tip has an application clearance angle of 15° in relation to the cutting plane. The chosen range of the application clearance angle, resulting from the configuration of the blade tip or blade cutting edge and by the blade reception angle of the blade holder, particularly advantageously permits the formation of the clearance space between the epithelium and the Bowman's membrane and thus the creation of the pre-tensioning of the connections between the epithelium and the Bowman's membrane. In addition to the advantages afforded by the thickness of the cutting edge and by the rounding of the cutting edge, the application clearance angle of the blade tip permits additional spreading of the clearance space and thus further contributes to creation of the pre-tensioning. During application of the blade, a lifting of the blade tip or cutting edge from the Bowman's membrane to be worked on is prevented by the application clearance angle of the blade tip. In this way, a collapse of the pre-tensioning is prevented when the open working angle between blade and Bowman's membrane drastically decreases as the blade passes the vertex of the eye.

The above-described blade holder, and a blade arranged removably therein, as described above, together form an ophthalmological device for cutting away an epithelial flap from a Bowman's membrane. For cutting away an epithelial flap from the Bowman's membrane, the blade to be inserted into the blade holder is preferably selected by first measuring the thickness of the epithelium and then choosing a blade which has a cutting edge with a thickness corresponding at least substantially to the measured thickness of the epithelium.

### BRIEF DESCRIPTION OF THE DRAWINGS

The present invention is described below on the basis of an illustrative embodiment. The illustrative embodiment is depicted in the following attached figures, where:

FIG. 1a shows a plan view of a blade for cutting away an epithelial flap from a Bowman's membrane, which blade comprises a blade tip with cutting edge, and also a blade shaft.

FIG. 1b shows a longitudinal section through the blade for cutting away an epithelial flap from a Bowman's membrane, which blade comprises a blade tip with cutting edge, and a blade shaft.

FIG. 2a shows a side view of a blade holder with a blade received therein in a removable manner, during cutting away of an epithelial flap from a Bowman's membrane.

FIG. 2b shows a detail of the blade holder from FIG. 2a, said detail showing an upward wall of the blade holder received in a recess of the blade tip upper face, and said detail also showing a cut-away epithelial flap lying on the blade tip upper face and on the upward wall.

FIG. 3 shows a detailed cross section depicting a blade tip inserted between the epithelium and the Bowman's membrane, the blade tip spreading open a clearance space between epithelium and Bowman's membrane.

FIG. 4a shows a cross section depicting a blade tip which has been inserted between the epithelium and the Bowman's membrane of an eye.

FIG. 4b shows a cross section depicting a blade tip which has cut away an epithelial flap from the Bowman's membrane of the eye as far as the vertex of the eye.

FIG. 4c shows a cross section depicting a blade tip which has cut away an epithelial flap from the Bowman's membrane of the eye beyond the vertex of the eye.

### WAYS OF IMPLEMENTING THE INVENTION

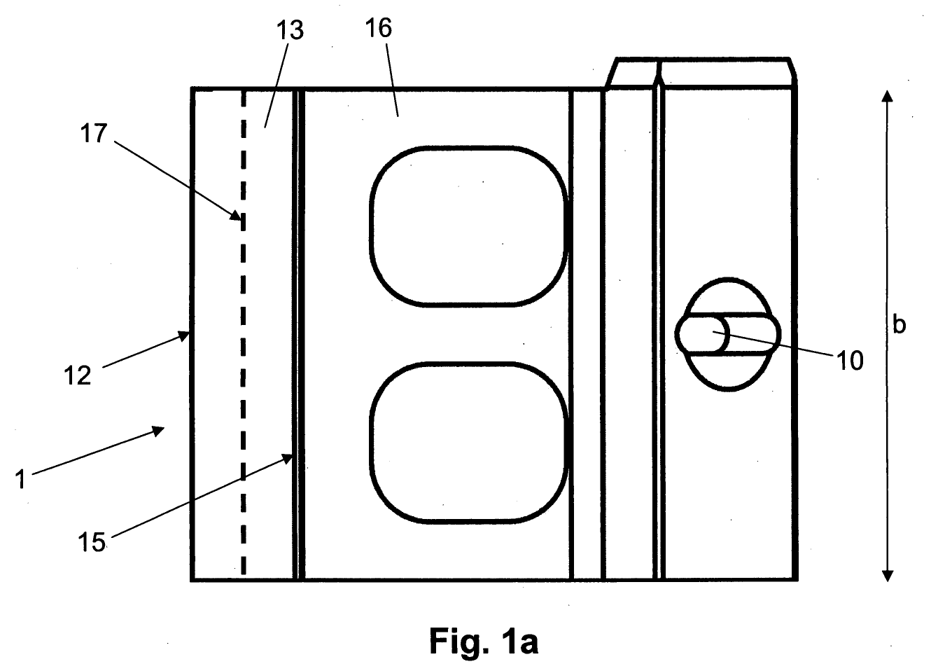

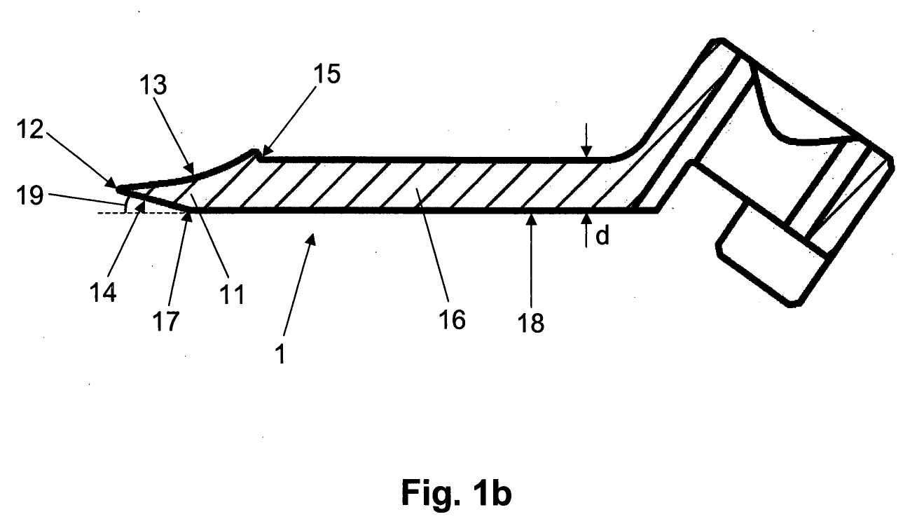

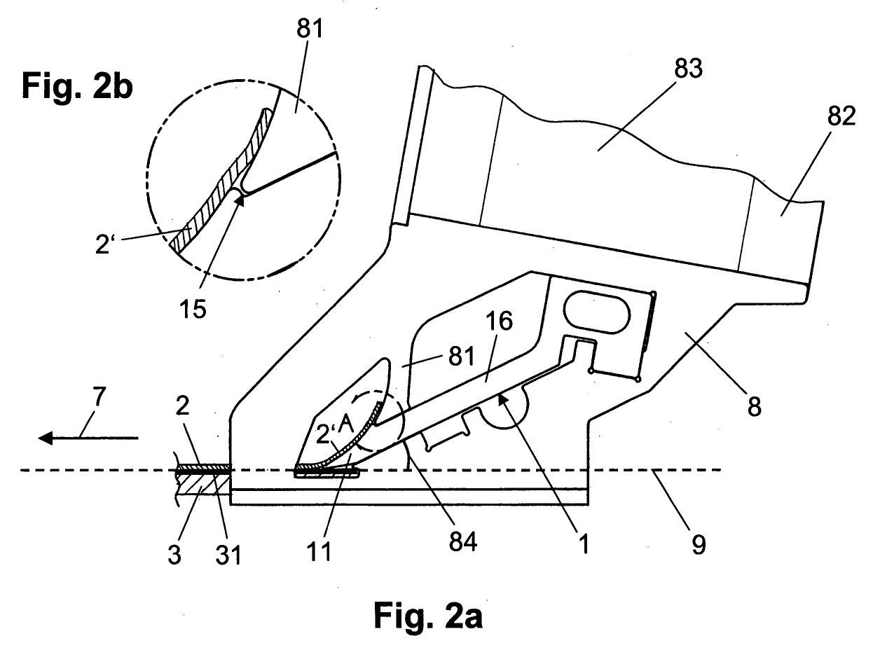

In FIGS. 1a, 1b and 2a, reference number 1 designates a blade for cutting away an epithelial flap from a Bowman's membrane. The blade is preferably produced in one part from plastic by injection moulding, for example from polycarbonate. As is shown in FIGS. 1a, 1b and 2a, the blade 1 comprises a blade tip 11 and a blade shaft 16. The blade 1 has a width b in the range of from 12 mm to 16 mm. The blade 1 has a thickness d in the range of from 0.5 mm to 2 mm. The cutting edge 12 of the blade 1 extends along the tapered end of the blade tip 11.

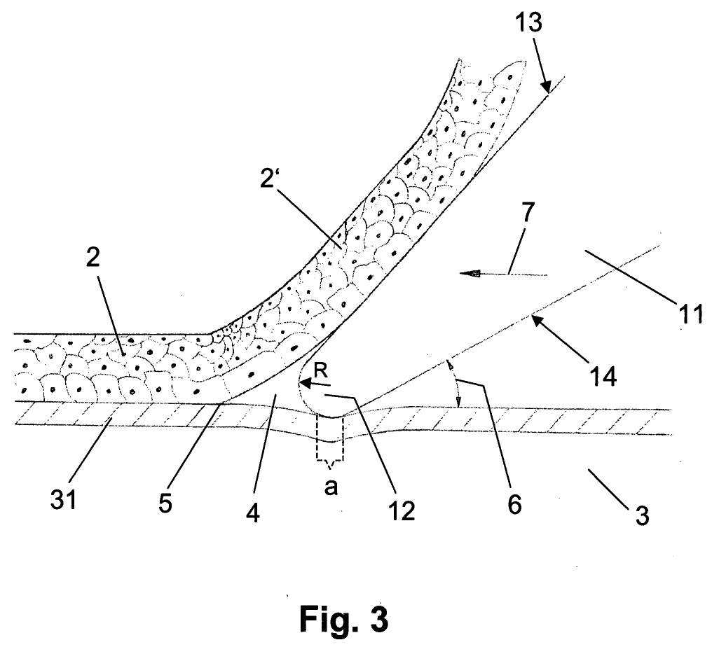

As is shown in FIG. 3, the cutting edge 12 is preferably rounded. The rounding of the cutting edge 12 has a radius R of at least half the thickness of the epithelium 2. For the cutting edge 12, there is therefore at least a thickness corresponding to the thickness of the epithelium 2. The radius R of the cutting edge is for example 30 μm or more, which gives a thickness of 60 μm or more for the cutting edge 12. The preferred thickness of the cutting edge 12 is in the range from one thickness of the epithelium 2 to one and a half thicknesses of the epithelium 2, that is to say in the range of from ca. 60 μm to 90 μm.

As can be seen in FIG. 1b, the blade 1 has a contact surface 14 which is arranged on the blade tip 11 and which is of an edgeless and smooth configuration. The contact surface 14 is angled away from the underside 18 of the blade shaft 16 starting from boundary line 17 and extends towards the cutting edge 12 in such a way that a clearance angle 19 of ca. 10° is obtained for the blade tip 11. The blade tip 11 additionally has a smoothly configured blade tip upper face 13 which is directed away from the contact surface 14 and extends towards the cutting edge 12. As can be seen from FIG. 1b, the contact surface 14 and the blade tip upper face 13 form a blade tip 11 cross section tapering towards the cutting edge 12. The length of the blade tip 11 from the cutting edge 12 to the boundary line 17 is ca. 1 mm to 2 mm.

In FIG. 2a, reference number 8 designates a blade holder. The blade holder 8 is designed to receive the blade 1 in a removable manner. For the removable connection of the blade 1 to the blade holder 8, the blade holder 8 comprises securing means (not shown). The securing means preferably comprise a plug connector. It is also possible, for example, to use a catch, clamping means or screws. The blade holder 8 additionally comprises diagrammatically illustrated drive means 82, 83, for example electric motors. The drive means 82 are designed to move the blade 1 in the advance direction 7, such that the cutting edge 12 is moved in the advance direction 7 in the cutting plane 9. The drive means 83 are designed to set the cutting edge 12 of the blade 1 in a lateral oscillating movement perpendicular to the advance direction 7 in the cutting plane 9. The drive means 83 are designed to generate the lateral oscillating movement with an oscillation stroke of the cutting edge 12 in the range of from 2 mm to 4 mm (oscillation amplitude of 1 mm to 2 mm), preferably ca. 3 mm (oscillation amplitude of ca. 1.5 mm), and with a frequency in the range of from 150 Hz to 200 Hz, preferably ca. 180 Hz. As is shown in FIG. 1a, the blade has a recess 10 in which a drive cam of the drive means 83 can be received for transmitting the oscillation movement.

The blade holder 8 is designed to receive the blade 1 in such a way that the blade tip 11 has an application clearance angle 6 in the range of from 10° to 30° in relation to the cutting plane 9. The application clearance angle 6 is preferably ca. 15°. The application clearance angle 6 of the blade tip 11 results from the blade reception angle 84 of the blade holder 8 (preferably 25°) by subtraction of the clearance angle 19 of the blade tip 11 (preferably 10°).

As can be seen from FIGS. 1b and 2a, the blade tip upper face 13 is preferably of concave design. In addition, the blade tip upper face 13 has a recess 15, in which recess 15 an upward wall 81 of the blade holder 8 can be received. As is shown in the detail in FIG. 2b, the concavely configured blade tip upper face 13 guides the cut-away epithelial flap 2′ smoothly, and without catching, across the gap which is formed between the blade tip upper face 13 and the upward wall 81 received in the recess 15. For this purpose, the recess 15 is dimensioned such that the blade tip upper face 13 protrudes slightly above the upward wall 81 at the transition to said upward wall 81.

In the detailed cross section in FIG. 3, the blade tip 11 is illustrated in the state of insertion between the epithelium 2 and the Bowman's membrane 31. The Bowman's membrane 31 is situated between the epithelium 2 and the stroma 3. It should be noted at this point that a basal membrane (not shown) is also situated between the epithelium 2 and the Bowman's membrane 31 and that the described separation of the epithelium 2 from the Bowman's membrane 31 can also include separation of the epithelium 2 from this basal membrane. As can be seen clearly from FIG. 3, in the state in which the blade tip 11 is inserted between the epithelium 2 and the Bowman's membrane 31, the blade tip 11 spreads open the clearance space 4 between the epithelium 2 and the Bowman's membrane 31. The clearance space 4 results in a pre-tensioning of connections between the epithelium 2 and the Bowman's membrane 31. The pre-tensioning is at its greatest at the apex 5 where the epithelium 2 and the Bowman's membrane 31 are still connected to one another. As is shown in FIG. 3, the blade tip 11 lies with an area a of the contact surface 14 at the cutting edge 12 on the Bowman's membrane 31 and exerts a slight pressure on the Bowman's membrane 31. In addition, the epithelium 2 is spread apart by the blade tip upper face 13. The pre-tensioning of the connections is generated in particular by the pressure exerted on the Bowman's membrane 31 and by the spreading apart of the epithelium 2.

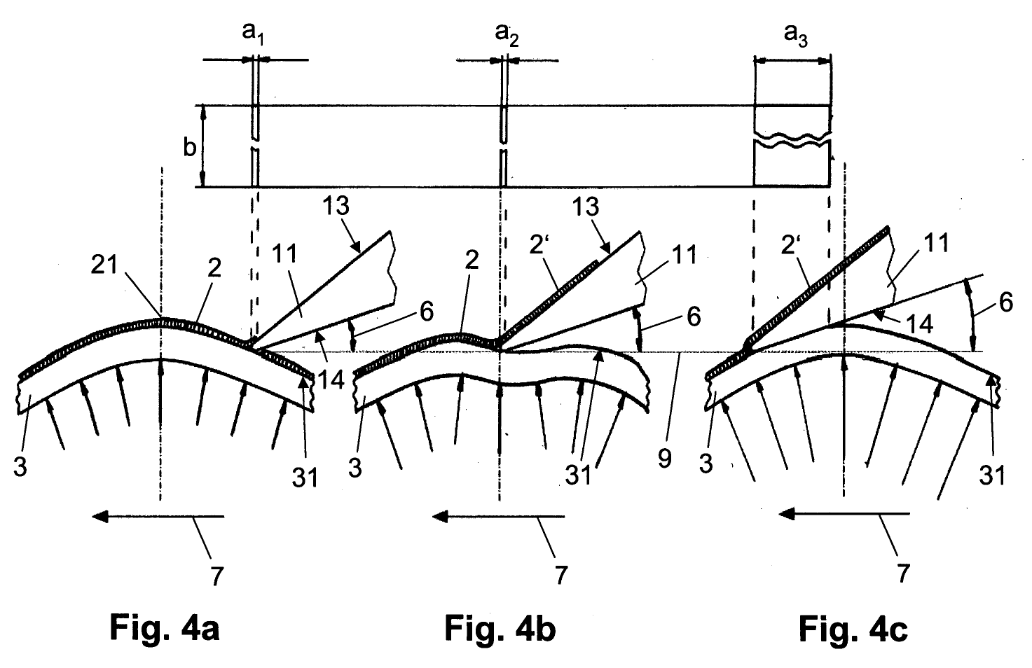

FIGS. 4a, 4b, 4c show diagrams illustrating different phases in cutting away the epithelium 2 from the Bowman's membrane 31. In FIGS. 4a, 4b, 4c, the Bowman's membrane 31 is shown only diagrammatically as a thin layer over the stroma 3. In FIG. 4a, the blade tip 11 is shown after piercing the epithelium 2, when the blade tip 11 is inserted between the epithelium 2 and the Bowman's membrane 31. As is shown in FIG. 4a, the blade tip 11 lies in the area a₁ on the Bowman's membrane 31, as has been explained above with reference to FIG. 3. The initially incised flap of the epithelium 2 slides on the blade tip upper face 13. In FIG. 4b, the blade tip 11 is shown on reaching the vertex 21 of the eye, when approximately half of the epithelial flap 2′ to be cut away has already been cut away from the Bowman's membrane 31. As is shown in FIG. 4b, the blade tip 11 lies in the area a₂ on the Bowman's membrane, and the epithelial flap 2′ cut away lies on the blade tip upper face 13 and is spread apart by the blade tip 11. The areas a₁ and a₂ correspond substantially to the area a of the contact surface 14 which was described above with reference to FIG. 3. In FIG. 4c, the blade tip 11 is shown after passing the vertex 21 of the eye, when the epithelial flap 2′ to be cut away has been cut away from the Bowman's membrane 31 almost to the planned extent. As is shown in FIG. 4c, the blade tip 11 lies in the area a₃ on the Bowman's membrane 31. The area a₃ is much larger than the areas a₁ and a₂ since the blade tip 11, after passing the vertex 21 of the eye, comes to lie with an increasingly larger part of the contact surface 14 on the Bowman's membrane 31 in the borderline case of the application clearance angle 6 shown in FIG. 4c. The application clearance angle 6, however, is preferably chosen such that the blade tip 11, even after passing the vertex 21 of the eye, does not lie with the area a₃ of the blade tip 11 but only with the area a (or a₁ and a₂) of the cutting edge 12 of the contact surface 14 on the Bowman's membrane 31. As long as the application clearance angle 6 is greater than 10°, this ensures that the blade tip 11 and in particular the cutting edge 12 does not lift from the Bowman's membrane 31 after passing the vertex 21 of the eye. In this way, the pre-tensioning of the connections between epithelium 2 and Bowman's membrane 31 can be maintained and collapse of the cutting pressure prevented.

1. Blade for cutting away an epithelial flap from a Bowman's membrane, comprising a blade shaft and, connected to the latter, a blade tip with cutting edge, wherein the blade tip is designed in such a way that, in the state in which the blade tip is inserted between epithelium and Bowman's membrane, said blade tip spreads open a clearance space between the epithelium and the Bowman's membrane, which clearance space results in a pre-tensioning of connections between the epithelium and the Bowman's membrane.

2. Blade according to claim 1, wherein the blade has an edgeless and smooth contact surface, and, in the state in which the blade tip is inserted between epithelium and Bowman's membrane, this contact surface presses at least partially on the Bowman's membrane.

3. Blade according to claim 2, wherein the contact surface is arranged on the blade tip, the contact surface being angled away from an underside of the blade shaft in such a way that the blade tip has a clearance angle.

4. Blade according to claim 1, wherein the cutting edge has a thickness which corresponds substantially at least to the thickness of the epithelium.

5. Blade according to claim 1, wherein the cutting edge has a thickness of at least 60 μm.

6. Blade according to claim 1, wherein the cutting edge is rounded.

7. Blade according to claim 1, wherein the blade tip has a smoothly configured blade tip upper face and, in the state in which the blade tip is inserted between epithelium and Bowman's membrane, the epithelial flap lies on this blade tip upper face, and in that the blade tip upper face has a recess, in which recess an upward wall of a blade holder can be received in such a way that the transition from the blade tip upper face to the upward wall does not form an obstacle to the epithelial flap sliding up it during continued cutting.

8. Blade holder designed to receive, in a removable manner, a blade according to claim 1.

9. Blade holder according to claim 8, wherein the blade holder comprises first drive means for moving the blade in an advance direction, and in that the blade holder comprises second drive means for setting the cutting edge of the blade in a lateral oscillating movement perpendicular to the advance direction in a cutting plane, a cumulative lateral travel of the cutting edge obtained from the oscillating movement being considerably greater than a travel of the cutting edge arising within the same time period in the advance direction.

10. Blade holder according to claim 9, wherein the second drive means are designed to generate the lateral oscillating movement with an oscillation stroke of the cutting edge in the range of from 2 mm to 4 mm.

11. Blade holder according to claim 9, wherein the second drive means are designed to generate the lateral oscillating movement with a frequency in the range of from 150 Hz to 200 Hz.

12. Blade holder according to claim 8, wherein the blade holder is designed to receive the blade in such a way that the blade tip has an application clearance angle in a range of from 10° to 30° in relation to a cutting plane.

13. Blade holder according to claim 8, wherein the blade holder is designed to receive the blade in such a way that the blade tip has an application clearance angle of 15° in relation to a cutting plane.

14. Method for selecting a blade according to claim 1 for cutting away an epithelial flap from a Bowman's membrane, comprising measuring the thickness of the epithelium and selecting the blade which has a cutting edge with a thickness corresponding substantially at least to the measured thickness of the epithelium.

15. Method for selecting a blade according to claim 2 for cutting away an epithelial flap from a Bowman's membrane, comprising measuring the thickness of the epithelium and selecting the blade which has a cutting edge with a thickness corresponding substantially at least to the measured thickness of the epithelium.

16. Method for selecting a blade according to claim 3 for cutting away an epithelial flap from a Bowman's membrane, comprising measuring the thickness of the epithelium and selecting the blade which has a cutting edge with a thickness corresponding substantially at least to the measured thickness of the epithelium.

17. Method for selecting a blade according to claim 4 for cutting away an epithelial flap from a Bowman's membrane, comprising measuring the thickness of the epithelium and selecting the blade which has a cutting edge with a thickness corresponding substantially at least to the measured thickness of the epithelium.

18. Method for selecting a blade according to claim 5 for cutting away an epithelial flap from a Bowman's membrane, comprising measuring the thickness of the epithelium and selecting the blade which has a cutting edge with a thickness corresponding substantially at least to the measured thickness of the epithelium.

19. Method for selecting a blade according to claim 6 for cutting away an epithelial flap from a Bowman's membrane, comprising measuring the thickness of the epithelium and selecting the blade which has a cutting edge with a thickness corresponding substantially at least to the measured thickness of the epithelium.

20. Method for selecting a blade according to claim 7 for cutting away an epithelial flap from a Bowman's membrane, comprising measuring the thickness of the epithelium and selecting the blade which has a cutting edge with a thickness corresponding substantially at least to the measured thickness of the epithelium.
